(19) United States
(12) Patent Application Publication
(10) Pub. No.: US 2020/0111548 A1
(43) Pub. Date: Apr. 9, 2020

(54) METHODS AND APPARATUSES TO VERIFY HOME HEALTH CARE

(71) Applicant: nVoq Incorporated, Boulder, CO (US)

(72) Inventors: Jon Ford, Timnath, CO (US); Charles Corfield, Boulder, CO (US)

(21) Appl. No.: 16/592,112

(22) Filed: Oct. 3, 2019

Related U.S. Application Data
(60) Provisional application No. 62/742,701, filed on Oct. 8, 2018.

(51) Int. Cl. G16H 10/60; G16H 40/20; G06F 21/32

(52) U.S. Cl. CPC G16H 10/60; G06Q 20/22; G06F 21/32; G16H 40/20

(57) ABSTRACT

A method, apparatus, or system for verifying the delivery of home health care is provided. The method, apparatus, or system includes obtaining information relating to at least one of a provider name, a location of treatment, a date of treatment, and a patient name from a billing application. Similar information is obtained from an electronic health record and/or data appended or associated with the electronic health record. A comparison between the billing application and electronic health record data provides data to verify the delivery of the home health care by the identified health care provider to the patient.

### CROSS-REFERENCE TO RELATED PATENT APPLICATIONS

This application claims priority to U.S. Provisional Patent Application Ser. No. 62/742,701, filed Oct. 8, 2018, the entire contents of which is incorporated herein by reference.

### BACKGROUND

Receiving healthcare at home, sometimes referred to as home healthcare or home care, provides supportive services in the home. Generally, a licensed healthcare provider renders medical services and/or products to a patient, which will generically be referred to as treatments. The healthcare provider may be a doctor (MD), a registered nurse (RN), a licensed nurse practitioner (LPN), a physician's assistant (PA), or other qualified person, which are generically referred to herein as provider. The provider renders treatments to the patient in the home. In many countries, the home care is paid for by a third party, such as, for example, a government or insurance company.

The growth of home care due to the aging population is creating a whole new set of challenges for the healthcare industry. Unlike in a clinic or hospital, the home provider is required to bring with them the supplies and equipment needed to provide treatment to the patient. And, the patient encounter is generally documented in an Electronic Health Records system both for ongoing quality of care as well as accurate and complete billing. The location and number of people involved with conventional care provides a self-auditing feature. Home care, on the other hand, often involves only a single provider of care and a patient at the patient's home. In these cases, accuracy and detail of the documentation are critical to the consistency and proof of care for billing, future medical reference, fraud prevention, and auditing. In addition to the patient encounter details, several data points provide future auditors with the information they need to verify that care was administered by the specified person, to the specified person, at the specified time and location.

In the more conventional patient/provider encounter at a clinic or hospital facility, the provider often dictates the interaction between the patient and the provider. The provider dictation may be for either human or computer transcription and the location of the patient and clinician are recorded as part of the medical record. Several individuals typically interact with the patient, creating a record of care that is auditable and sufficient for future reference whether for billing or ongoing treatment. For the purposes of this application, the terms electronic health record, health record, medical record, and record are generally used interchangeably.

Home healthcare, on the other hand, often involves only a single provider of care and a patient. In these cases, accuracy and detail of the documentation are critical to the consistency and proof of care for billing, future medical reference, fraud prevention, and auditing. This is especially true as often the patient encounter is not witnessed. Although the patient encounter is documented in the record, additional data points may be useful to provide future auditors with the information they need to verify that care was administered by the specified provider, to the specified patient, at the specified time and location.

Against this background, it would be desirous to provide methods and apparatuses that can verify the home care visits, provisions, and services.

### SUMMARY

This Summary is provided to introduce a selection of concepts in a simplified form that are further described below in the Detailed Description. This Summary, and the foregoing Background, is not intended to identify key aspects or essential aspects of the claimed subject matter. Moreover, this Summary is not intended for use as an aid in determining the scope of the claimed subject matter.

In one objective of the technology, a method of verifying the delivery of home care treatment to a patient is provided. The method includes extracting, by a processor, data from an electronic medical billing application including at least a provider name, a location of treatment, a date of treatment, and a patient name. Extracting, by a processor data from a health record application including at least a provider name, a location of treatment, a date of treatment, and a patient name. Obtaining biometric information from the health record application identifying the provider of the treatment. Determining whether the data extracted from the electronic medical billing application matches the data extracted from the health record application. Finally, verifying the biometric information obtained from the provider of the treatment matches a verification biometric information stored in the health record application for the identified provider. In certain embodiments, the health record application stores at least an encounter audio file from the provider wherein the biometric information obtained from the provider comprises a voice print from the encounter audio file and the verification biometric information is a voice print from a verification audio file. Additionally, in certain embodiments, the method includes verifying the biometric information obtained from a patient during the provision of treatment by the provider matches a verification biometric information stored in the health record application for the patient.

In certain aspects, the biometric information may be a voice print established from audio recordings or developed in real time from an audio sample. In other aspects, the biometric information may be, among other things, an iris scan, a retinal scan, a fingerprint, facial recognition, DNA, other chemical recognition, a combination thereof, or the like.

In another objective of the technology, a method of providing an auditable record for the delivery of home care by a provider to a patient is provided. The method comprises invoking a health record application on a smart device by a provider of home care and identifying a provider using a unique identification to access the health record application. The provider enters data to the health record application via a user interface on the smart device. The method also determines a date, time and location for the entry of the data. The provider is verified with biometric data stored in the health record application and verification biometric data and the health record application provides a link of the provider identification, date, time, and location to the data stored in the health record application wherein the provider identification, date, time, and location can be verified.

In still further aspects, embodiments of the technology of the present application may include:

1. A method, apparatus, or system of verifying the delivery of home care treatment to a patient, comprising extracting, by a processor, data from an electronic medical billing application including at least a provider name, a location of treatment, a date of treatment, and a patient name; extracting, by a processor data from a health record application including at least a provider name, a location of treatment, a date of treatment, and a patient name; obtaining biometric information from the health record application identifying the provider of the treatment; determining whether the data extracted from the electronic medical billing application matches the data extracted from the health record application; and verifying the biometric information obtained from the provider of the treatment matches a verification biometric information stored in the health record application for the identified provider.

2. The method, apparatus, or system of embodiment 1 wherein the health record application stores at least an encounter audio file from the provider.

3. The method, apparatus, or system of any or the preceding embodiments wherein the biometric information obtained from the provider comprises a voice print from the encounter audio file and the verification biometric information is a voice print from a verification audio file.

4. The method, apparatus, or system of any or the preceding embodiments further comprising: verifying the biometric information obtained from a patient during the provision of treatment by the provider matches a verification biometric information stored in the health record application for the patient.

5. The method, apparatus, or system of any or the preceding embodiments wherein the biometric information obtained from the patient is a confirmation receipt audio file from which a voice print is obtained and the verification biometric information for the patient is a verification audio file from which a voice print is obtained.

6. The method, apparatus, or system of any or the preceding embodiments wherein data extracted from the electronic medical billing application includes a classification code, and the method further comprises determining whether the classification code matches the health record application data.

7. A method, apparatus, or system of providing an auditable record for the delivery of home care by a provider to a patient, comprising invoking a health record application on a smart device by a provider of home care; identifying a provider using a unique identification to access the health record application; entering data to the health record application via a user interface on the smart device; determining a date and time that the data is entered into the health record application determining a location of the smart device using a location-based module; and verifying the provider with biometric data stored in the health record application and verification biometric data; and linking the provider identification, date, time, and location to the data stored in the health record application wherein the provider identification, date, time, and location can be verified.

8. The method, apparatus, or system of any or the preceding embodiments wherein the location-based module comprises satellite navigation.

9. The method, apparatus, or system of any or the preceding embodiments wherein the data entered to the health record application comprises an encounter audio file comprising notes.

10. The method, apparatus, or system of any or the preceding embodiments wherein the biometric data comprises a voice print from the encounter audio file and the verification biometric data comprises a voice print from a verification audio file.

11. The method, apparatus, or system of any or the preceding embodiments wherein the data entered to the health record application comprises text generated from the encounter audio file.

12. The method, apparatus, or system of any or the preceding embodiments wherein the health record application comprises an electronic health record.

13. A method, apparatus, or system of verifying the delivery of goods or services to a customer, comprising extracting, by a processor, data from an electronic billing application including at least a provider name, a location of the delivery, a date of the delivery, and a customer name; extracting, by a processor data from a delivery application including at least a provider name, a location of the delivery, a date of the delivery, and a customer name; obtaining biometric information from the delivery application identifying the provider of the delivery; determining whether the data extracted from the electronic billing application matches the data extracted from the delivery application; and verifying the biometric information obtained from the provider of the delivery matches a verification biometric information stored in the delivery application for the identified provider.

These and other aspects of the present system and method will be apparent after consideration of the Detailed Description and Figures herein.

### DRAWINGS

Non-limiting and non-exhaustive embodiments of the present invention, including the preferred embodiment, are described with reference to the following figures, wherein like reference numerals refer to like parts throughout the various views unless otherwise specified.

FIG. 1 is a diagrammatic representation of a home care provider/patient encounter consistent with the technology of the present application.

FIG. 2 is a flow diagram representative of appending information to an audio file associated with the provider/patient encounter consistent with the technology of the present application.

FIG. 3 is a flow diagram representative of verifying information consistent with the technology of the present application.

FIG. 4 is a flow diagram representative of verifying information consistent with the technology of the present application.

FIG. 5 is a flow diagram representative of verifying information consistent with the technology of the present application.

FIG. 6 is a schematic diagram of a machine consistent with the technology of the present application.

### DETAILED DESCRIPTION

The technology of the present application will now be described more fully below with reference to the accompanying figures, which form a part hereof and show, by way of illustration, specific exemplary embodiments. These embodiments are disclosed in sufficient detail to enable those skilled in the art to practice the technology of the present application. However, embodiments may be implemented in many different forms and should not be construed as being limited to the embodiments set forth herein. The following detailed description is, therefore, not to be taken in a limiting sense.

The technology of the present application is described with specific reference to providing home care to patients by a provider. The specific example may include reference to a provider rendering service to a patient, but the technology described herein also relates to the provision of goods as well as services. The delivery of medical goods and/or services will generally be referred to generically as treatment. The technology described herein relates, in part, to the problem of verifying home care. Thus, the technology generating a storable data set or file having additional data points to facilitate verification of the encounter between the provider and the patient. However, the technology described herein may be used for recognizing other types of services, such as, for example, accounting services, legal services, appliance repair services, car repair services, physical therapy (outside of home care), telecommunication services, etc. to name but a few other types of activities that may require a storable data set of a service or good delivery that is capable of being audited and/or verified. In certain aspects, the technology will identify a location of the encounter, verify the provider, and verify the patient (or recipient) to name but a few concepts to facilitate auditing of a record.

The technology of the present application will be described with relation to exemplary embodiments. The word “exemplary” is used herein to mean “serving as an example, instance, or illustration.” Any embodiment described herein as “exemplary” is not necessarily to be construed as preferred or advantageous over other embodiments. Additionally, unless specifically identified otherwise, all embodiments described herein should be considered exemplary.

The technology described herein relates to verifying the delivery of goods and services, also known as treatment, by a provider to a patient during a home care visit. Verify in this instance means, among other things, providing data points to facilitate the auditing of one or more invoices relating to the treatment. Home care, unlike more traditional care, is frequently given by one provider with one patient at the patient's home (or other facility associated with the patient). Thus, it is difficult to confirm the delivery of services should a 3rd party payor desire to audit the one or more invoices or should a patient dispute an item on the one or more invoices. Additionally, methods and apparatuses to verify the delivery of treatment is important for accuracy and detail as the information is critical for future medical reference by providers.

As can be appreciated, most home care providers in developed countries use some type of device to document a patient encounter. The device typically allows for manual entry or dictation to the electronic medical record or health record application, which may be an Electronic Medical Record or similarly defined record or database of relevant data. When the goods or services are not treatments as defined above, the health record application may be a delivery application or the like containing the relevant information and data points. The device to record the dictation, or data otherwise manually entered, may be a non-internet enabled or an internet enabled device. Internet enabled devices are generally referred to as smart devices. Smart devices are generally ubiquitous in most developed countries. As used herein, a smart device may include, for example, an internet enabled cellular phone (including an iPhone, a Galaxy, a Note, a Pixel, or the like), an internet enabled tablet (such as an iPad, a Surface, a Kindle, or the like), a laptop computer, an Internet of Things enable device, or the like.

Smart devices, and some non-internet enabled devices, include at least one location-based module associated with the device. Generally, the location-based module will access a satellite navigation system to identify the location of the smart device at any given time. Global satellite navigation systems include GPS—US, GLONASS—Russian, Galileo—Europe, and BDS—China, etc. Satellite navigation systems generically refers to any satellite-based system to determine the geo-spatial position of the device. In many instances, the satellite-based navigation systems can be used to identify the location of the device. Often satellite-based navigation systems are coupled with a mapping application such that the geospatial location from a satellite-based navigation can be converted to a more conventional street address. Other types of location-based modules are usable to detect the location of a device including, for example, triangulation methods using cellular towers, location-based services providing by an operating system, WiFi based location services, etc. or a combination thereof. The location-based module, in many instances, will map the geo-spatial location to a specific street address. For multi-dwelling units, such as apartments, the location-based service may include a combination of a satellite navigation system (for the street address, for example) and a WiFi based location service for the floor or unit number of the multi-dwelling unit. Additionally, multiple location-based services may be available for any smart device at any location. In certain instances, the most accurate location-based service may be used by the smart device. For purposes of this application, location or location-based generally refers to both geospatial and street address. However, for the specific purpose of verifying home care, the invoicing is traditionally associated with a street address.

The smart device would be configured with a health record application. In certain embodiments, the health record application on the smart device would be a thin client device, which essentially provides a user interface to a remote server hosting the health record application. In other embodiments, the health record application is hosted on the smart device, which would be a thick client. Storing data or notes associated with the health record application may mean writing data directly to the device, batch transferring data from the device to a remote server, streaming data from the device to the remote server, a combination thereof or the like. The health record application may include, among other things, a database or the like to organize the data stored by the health record application.

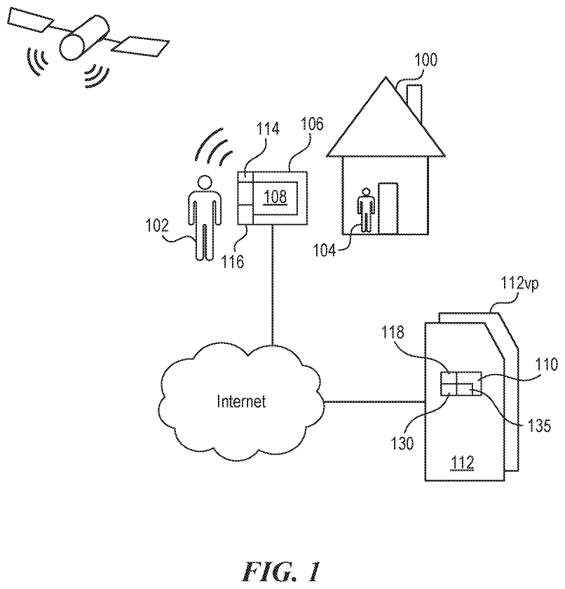

FIG. 1 provides a rendering of an interaction between a provider 102 and a patient 104 in a home 100. The provider 102, in this exemplary embodiment, has a smart device 106. The smart device 106 has a user interface 108 that interacts with a health record application 110. As shown, the smart device 106 is a thin client, in this exemplary embodiment, so portions of the health record application 110 may reside on a server 112 that is remote from the smart device 106. As mentioned above, the smart device 106 includes a location-based module 114 to identify the street address (or the geospatial location which is converted to a street address) of the smart device 106.

During the provider/patient encounter, the provider 102 will enter information to the health record application 110 through the user interface 108. The information transmitted to the health record application 110 (or saved to the health record application 110 if a thick client) is appended with the street address determined by the location-based module 114. Additionally, the date and time is appended to the data.

As mentioned above, in one aspect to the technology, the provider may dictate the provider/patient encounter for the health record application 110. The dictation provided to the health record application 110 is transcribed and input to an electronic medical record. The transcription may be by a service or automated by a speech processor. When configured to receive dictation, the health record application 110 is capable of receiving and storing an audio file associated with the provider/patient encounter. Thus, the provider 102 may dictate using a microphone 116 connected to the smart device 106 (which may be the cell phone microphone or the like) to generate an audio file, which will be identified herein as the encounter audio file 118. The encounter audio file 118 containing the notes from the provider/patient encounter would be stored by the health record application 110 on the server 112 in this exemplary embodiment. The smart device 106 appends the encounter audio file 118 with the date, location, and time data. The appended location, date, and time data may be contained in a metadata field or other data field associated with the encounter audio file 118. Alternatively, the location and time data may be saved in a separate file with a link to the location and time file stored in the database. The location and time data may be encrypted, hashed, or otherwise protected from tampering. In certain embodiments, the location and time data may be separately transmitted to the health record application 110 for storage. In some aspects, there may be multiple transmissions or recordings of location and time data including, for example, the location, time, and date data for when the encounter audio file 118 is started and the location, time, and date data for when the encounter audio file 118 is ended.

As can be appreciated, appending the location, time, and datetime date of the encounter audio file 118, or linking the location, time, and date file to the encounter audio file 118, provides an important data point for auditing or verifying the provider/patient encounter for home care. Specifically, the street address (whether a single-family unit or a multiple-family unit) for the home care is a known data point. Additionally, the date and time for the delivery of the home care is known from a scheduling and/or dispatch application. Thus, when auditing or verifying the provider/patient encounter, the auditor can compare the location, time, and date of the generation of the encounter audio file 118 with the known location, time, and date for the home care visit to verify the encounter audio file 118 was generated at the correct location, time, and date or with a predefined accuracy. Accuracy, for the present application means, among other things, that the location is within a predefined distance and the encounter audio time and date is within a predefined amount of time and date from the known (or prearranged) encounter time and date.

Importantly for home care, the encounter audio file 118 and the saving, transmission, and security of the encounter audio file 118, as well as any data, must comply with HIPPA requirements and protocols. Also, while the exemplary embodiment provides for home care, other home services that may be paid by a 3rd party may use the technology herein without having the same HIPPA security and protection protocols.

The health record application 110 typically would associate a unique identification for the provider inputting the notes to the health record application 110. Thus, when the health record application 110 is invoked on the smart device 106, the health record application 110 associates the session with a provider account. Thus, the unique identification would be associated with the provider/patient encounter notes, such as, for example, the encounter audio file 118. Similar to the location, time, and date data, the unique identification for the provider may be appended to the encounter audio file 118, such as in the metadata, or the unique identification may be stored separately with a link between the unique identification and the encounter audio file 118. When the health record application 110 includes a dictation component such that encounter audio file 118 is generated, the following information may be linked or stored in a database for verification/auditing of the provider/patient encounter: the encounter audio file 118, any associated text generated by the encounter audio file 118 (including an electronic health record if one is applicable), the location of the device used to generate the audio file 118, the time date that the audio file 118 was generated, and the provider's unique identification.

While useful from an auditing, verification perspective, having the provider's unique identification, the location and time information, and audio and text of the provider/patient encounter may not be sufficient information in all auditing or verification situations. For example, the provider's unique identification is evidence that the user login was provided, but it does not confirm that the person dictating, or manually entering data, is, in fact, the provider. Additionally, the provider may have dictated the audio file while located on the doorstep of the patient without actually providing the home care. Thus, the technology of the present application provides a mechanism to authenticate the provider and authenticate the receipt of the treatment by the patient.

With the above in mind, the encounter audio file 118 provides information that may be useful in identifying or confirming the person dictating the encounter audio file 118 is the same person associated with the provider' unique identification, which unique identification may be conventional login credentials. To provide speaker verification, the provider will have verification audio file (or files) 130 stored. The verification audio file 130 is linked to the provider's health record application 110 account. The verification audio file 130 may be other encounter audio files related to other provider/patient encounters, predefined text dictated by the provider, a combination thereof, other information, or the like. The verification audio file 130 may, in certain aspects, be data converted into a stable signature where the false positive and negative rates of identification are within an acceptable tolerance or threshold. For example, audio may be used to generate a conventional voice print where the voice print is stored as the verification audio file 130. The server 112 hosting the health record application 110 (which may be a local or remote processor) compares voice prints from the verification audio file 130 and the encounter audio file 118 to confirm whether the person who dictated the encounter audio file 118 is the person who generated the verification audio file 130. The recognition may compare the voice prints using, among other things, frequency analysis and power analysis consistent with known speaker verification techniques to confirm the encounter audio file 118 generated at the location, time, and date is the same as the person who generated the verification audio file 130.

Moreover, to confirm receipt of treatment, the provider may request the patient to verbally confirm receipt of the treatment and record the receipt confirmation audio file 135. The receipt confirmation audio file 135 would be recorded using the microphone 116 of the smart device 106. The receipt confirmation audio file 135 would be linked or appended to the encounter audio file 118, along with the date, time, and location information, and stored with the health record application 110 (or in the associated database that provides links between the files). The verbal receipt confirmation, also known here as the receipt confirmation audio file 135, provides a data point to verify the provision of treatment by the provider to the patient. The technology of the present application contemplates verifying the patient information in a number of different ways. For example, the patient may be required to provide a patient verification file when enrolling for the provision of home care to allow for a voice print comparison between the patient verification file and the receipt confirmation audio file 135. In another embodiment, the patient may be required to provide a verification audio file as part of an audit or verification process. Thus, the health record application 110 may store verification files for the patient as well as the provider. Using the voice prints, the delivery of the treatment by the provider can be verified by the voice print and the receipt of the treatment by the patient can be provided.

The present exemplary embodiment of the technology of the present application provides a health care application that receives dictated notes from the provider/patient encounter via the transmission or storage of the encounter audio file 118. In certain embodiments, however, the health record application 110 may not receive audio files. Rather, as outlined above, the provider may enter data manually via the user interface 108. The data file written to memory by the health record application 110 may be linked to the provider's unique identification information along with the location, date, and time of the creation of the data file. Linking the location, date, and time to the data file may occur using methodologies similar to those outlined above. The data file along with the provider's identification, location, date, and time may be sufficient in a majority of cases to verify delivery of treatment during the provider/patent encounter. However, rather than a voice print verification, other biometric information may be provided appended to the data file. For example, based on manual data entry through the user interface 108, the data will be uploaded to the health record application 110 and written to a memory, for example, in a relationship database. Similar to storing verification audio files, the health record application 110 may store in memory, such as in a database format or the like, the provider's biometric information. Such information may be requested at the time of account setup and the biometric information is linked to the login credentials, or unique identification. At some point during the provider/patient encounter, the provider, and the patient, in certain embodiments, may be requested to provide a fingerprint, retinal scan, or other biometric identifier to confirm the identity. Similarly, the biometric identifier for the provider, and the patient, in certain embodiments, may be linked to the health record application 110. For example, when first establishing a provider account and login for the health record application 110, the provider may be required to provide verification biometric data. The biometric identifier provided with the writing of the provider/patient data by the health record application 110 to memory may be compared to the verification biometric data already stored to verify the provider. Similarly, when the patient registers for home care, the patient may be required to provide biometric information, such as a fingerprint or the like.

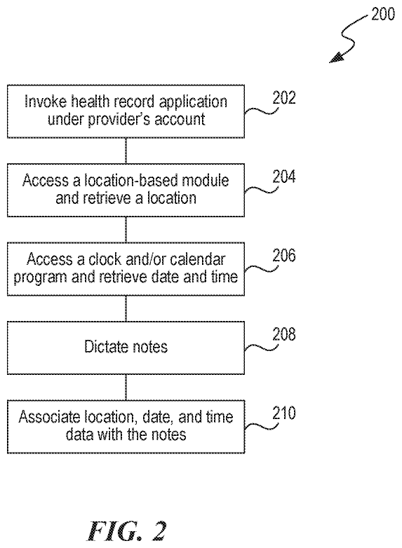

FIG. 2 provides an exemplary flow diagram 200 of a method of creating a record of the provider/patient encounter consistent with the technology of the present application. To begin the delivery of home care, the provider invokes a health record application 110 on a smart device 106, step 202. The invoked health record application 110 will be linked to a provider's unique identification. While the health record application 110 is invoked, the smart device 106 accesses one or more location-based modules to retrieve a location of the device when the health record application 110 is invoked, step 204. Similarly, the smart device 106 accesses a clock and data module to record the time and date, step 206. Location may mean a street-based address, such as a mailing address, or a geospatial location. In this exemplary embodiment, the provider accesses the microphone 116 of the smart device 106 to dictate notes of the provider/patient encounter that are saved as an encounter audio file 118, step 208. The encounter audio file 118 may be saved to the smart device 106, batch uploaded from the smart device 106 to the server 112, or streamed to the server 112, through a communication link, such as the internet, wired or wireless connections, or a cellular link. Wherever saved, the encounter audio file 118 is associated with the location, date, and time information, step 210. The association may be by saving the location, date, and time information in separate files and providing a link between encounter audio file 118 and the date, location, and time information files or by appending the encounter audio file 118 with the location, time, and date information via, for example, the metadata of encounter audio file 118.

The encounter audio file 118 generated in connection with the health record application 110 to document the provider/patient encounter includes links and ties between the encounter audio file, the provider's unique identification, the location, the date, and the time of the provider patient encounter. In certain embodiments, the encounter audio file (any transcripts thereof), the unique identification of the provider, and the time, date, and location data are sufficient to verify treatment provided by the provider to the patient.

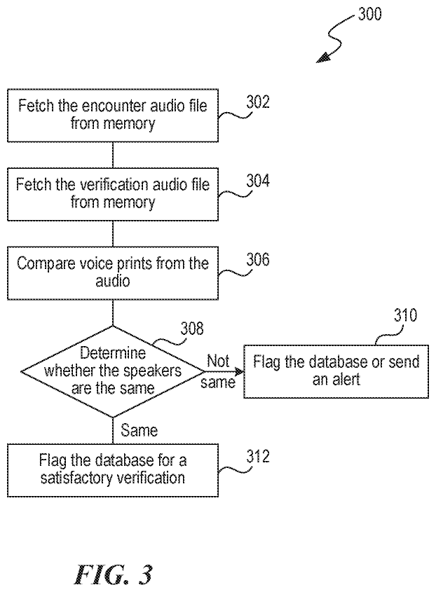

In certain aspects, however, confirmation that the provider who generated the audio file 118 via dictation is the same person as who established the unique identification for the health record application 110 is required. In this case, the server 112 (or smart device 106) may provide a voice print authentication. A voice print authentication is generally known in the art, but compares, in this exemplary embodiment, the encounter audio file 118 and verification audio file 130. FIG. 3 shows one exemplary flow diagram 300 for the voice print authentication. First, a processor, such as server 112, fetches the encounter audio file 118 of the dictated notes relating to the provider/patient encounter, step 302. Next, at step 304, the processor, such as server 112, fetches the verification audio file 130 already stored. The verification audio file 130 may be predefined text or simply other audio from the speaker. A voice print authentication engine, such as a voice print authentication engine 112vp associated with server 112, compares the audio file 118 and the verification audio 130 to confirm whether the speaker for the audio file 118 is the same as the speaker for the verification audio 130, which confirms that the provider with the unique identification, in fact, dictated the encounter audio file 118, step 306. In many instances, the authentication will be within a tolerance, such as, for example, 90% likelihood that the speaker is verified as the provider. Next, it is determined whether the speaker for the encounter audio file and the verification audio is the same, step 308. If the speakers are not verified as the same, the processor may send an alert or tag the database storing the information, step 310. If the speakers are verified as the same, the processor may tag the database that the verification was satisfactory, step 312

The verification process above can be carried out during a verification or audit of the provider/patient encounter during home care visit. Alternatively, the verification process can be completed prior to the verification or audit and the result appended to the data in the health record application 110 of the visit.

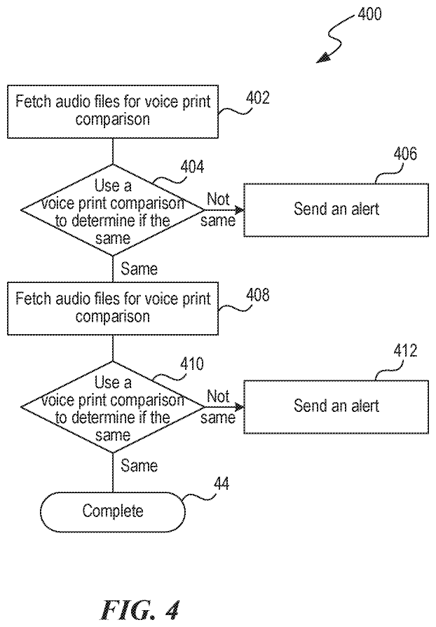

In certain embodiments, the verification process can be automated. FIG. 4 shows an exemplary flow diagram of a methodology 400 associated with verifying the provider/patient encounter when the provider dictates the encounter using encounter audio file 118 and the patient verbally confirms the receipt in the same encounter audio file 118 or in a receipt encounter audio file. First, the processor, whether the device 106 processor or a server 112 processor fetches the encounter audio file 118 and verification audio file 130, step 402. Using conventional voice print technology, the processor would determine whether the voice prints from the encounter audio file 118 and the verification audio file 130 are from the same as the speaker that dictated the verification audio file 130, step 404. The verification may be based on verification within a certain confidence level, such as, for example, 90% likely. If it is determined they are not the same, the process stops and provides an alert, step 406. Otherwise, the process continues with the patient verification. The patient verification would be similar should the patient provide audio for a receipt of the treatment as shown in steps 408 and 410. The patient verification audio may be pre-recorded when the patient enrolls for home care or at the time of an audit or protest. If the patient verification fails, the process provides an alert, step 412. Otherwise, the verification process is complete, step 414. If audio is not available, the verification process can use alternative forms of biometric information, such as, for example, fingerprints and the like.

In certain aspects, the verification process further can confirm the home care treatment by comparison of the invoice (electronic billing records) and the medical records. As mentioned above, the encounter audio file 118 is a dictation file of the encounter notes. The dictation is transcribed and saved as part of the health record application 110 data. Typically, in the United States, for example, the provider/patient encounter is submitted for payment using a conventional electronic medical billing system. The invoice process uses numerical codes, such as from the International Classification of Diseases (ICD) as revised from time to time, that are electronically submitted to the payor of the home care, which may be a government or an insurance company. The transcription file stored with the encounter audio file 118 can be scanned for keywords associated with the ICD billing information to verify the invoicing and the treatment are the same. Thus, for example, the provider may dictate an elevated blood-pressure reading as part of the encounter audio file 118, which would be entered into the health record application 110 as part of the electronic health record for the patient. The billing specialist for the practice, however, may code the invoice as R03.1 that is the billable code for a nonspecific low blood-pressure reading. During the verification step, the invoice code R03.1 may generate key words of low, blood, and pressure. The processor may compare the transcript of the patient encounter for the keyword or keywords, which would be elevated, blood, and pressure in this example. Thus, the comparison would indicate false as the keyword low would not be found.

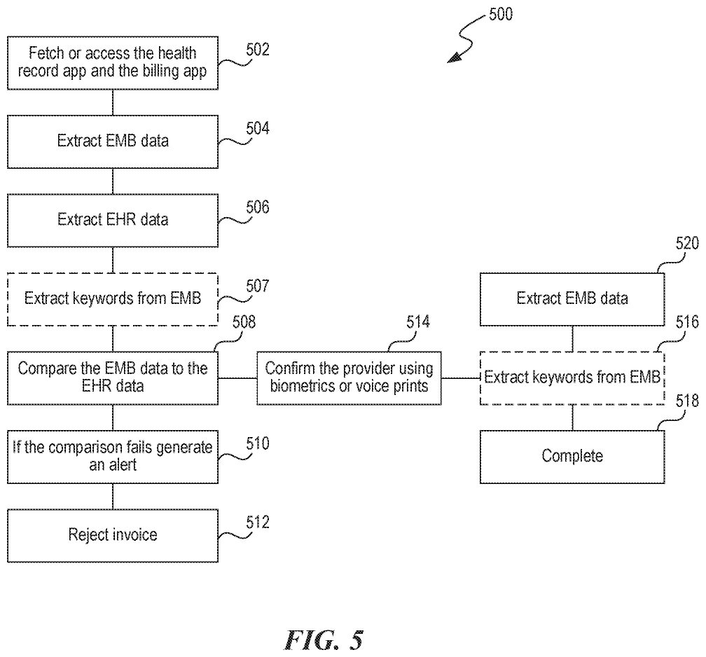

In certain aspects, the verification process may be automated by the present technology. FIG. 5 shows a verification methodology 500 consistent with the technology of the present application. The verification process starts by fetching or accessing the health record application 110 associated with the provider/patient encounter as well as by fetching or accessing the electronic medical billing application associated with the provider/patient encounter, step 502. Next, the processor extracts from the electronic medical billing information data (EMB data) to verify/audit the invoice, step 504. The data may include, among other things, the location, time and date of the delivery of treatment, the provider identification that delivered the treatment, the patient identification that received the treatment, the ICD code, and the like. The processor also extracts from the health record application 110, the provider identification (associated with the unique identification), the patient's identification (if available), and the location, time and date information linked or appended to the encounter audio file, (a.k.a. EHR data), step 506. For purposes of the present technology, steps 504 and 506 may be reversed or occur substantially simultaneously. Optionally, the processor may extract key words associated with the EMB from the ICD code as part of step 504 or as a separate step 507. The processor next compares the extracted information from the health record application 110 and the electronic medical billing information, step 508. The comparison compares, among other things, whether the encounter audio file 118 location is within a predefined distance of the street address indicated in the electronic medical billing information. The comparison may be done for all or a subset of the provider identification, the patient identification, the date, the time, etc.

If any of the above information does not stand up to the comparison, the verification processor may generate an alert, step 510. The alert may require a manual review of the invoice and provider/patient encounter. The alert may cause the electronic medical billing invoice to be rejected, step 512. Otherwise, the process continues.

In certain embodiments, the above verification may be sufficient for the process of the present application. However, in certain embodiments, additional verification may be desirable. Thus, assuming the time, date, location, provider user identification, and patient user identification are sufficient, the process may continue by comparing a voice print from the encounter audio file 118 with a voice print from the verification audio file 130 for the provider, step 514. Assuming the voice print verifies the provider, the processor optionally may confirm the patient by performing a voice print of patient audio, step 516. Of course, other biometric information can be substituted for the voice print. If the verification by voice print (or other biometrics) is successful, the audit is complete, step 518. Otherwise, if the verification by voice print (or other biometric) is not successful, an alert is sent, step 520, which may result in a manual investigation.

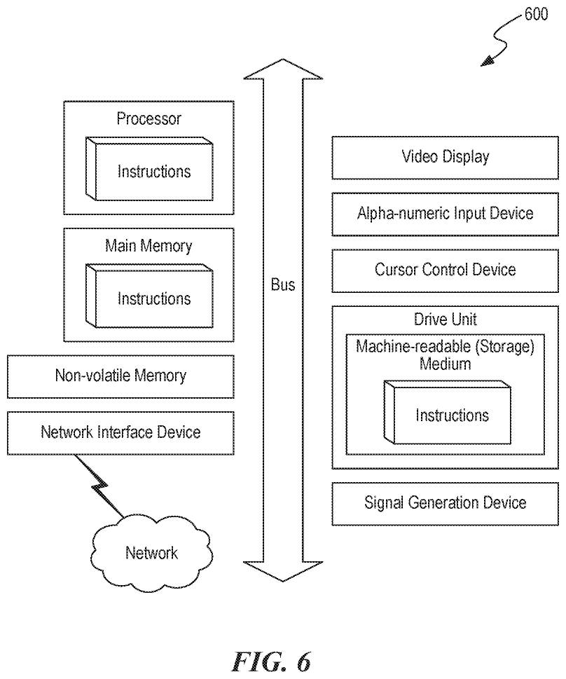

The technology described herein optionally comprises many networked machines. FIG. 6 depicts a diagrammatic representation of a machine, in the example form, of a computer system within which a set of instructions, for causing the machine to perform any one or more of the methodologies discussed herein, may be executed.

In the example of FIG. 6, the computer system 600 includes a processor, memory, non-volatile memory, and an interface device. Various common components (e.g., cache memory) are omitted for illustrative simplicity. The computer system 600 is intended to illustrate a hardware device on which any of the functions, applications, engines, and scripts are running as described herein and shown in figures (and any other components described in this specification) can be implemented. The computer system 600 can be of any applicable known or convenient type. The components of the computer system 600 can be coupled together via a bus or through some other known or convenient device.

The processor may be, for example, a conventional microprocessor such as an Intel microprocessor, Motorola microprocessor, or the like. One of skill in the relevant art will recognize that the terms “machine-readable (storage) medium” or “computer-readable (storage) medium” include any type of device that is accessible by the processor.

The memory is coupled to the processor by, for example, a bus. The memory can include, by way of example but not limitation, random access memory (RAM), such as dynamic RAM (DRAM) and static RAM (SRAM). The memory can be local, remote, or distributed.

The bus also couples the processor to the non-volatile memory and drive unit. The non-volatile memory is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software in the computer 600. The non-volatile storage can be local, remote, or distributed. The non-volatile memory is optional because systems can be created with all applicable data available in memory. A typical computer system will usually include at least a processor, memory, and a device (e.g., a bus) coupling the memory to the processor.

Software is typically stored in the non-volatile memory and/or the drive unit. Indeed, for large programs, it may not even be possible to store the entire program in the memory. Nevertheless, it should be understood that for software to run, if necessary, it is moved to a computer readable location appropriate for processing, and for illustrative purposes, that location is referred to as the memory in this paper. Even when software is moved to the memory for execution, the processor will typically make use of hardware registers to store values associated with the software, and local cache that, ideally, serves to speed up execution. As used herein, a software program is assumed to be stored at any known or convenient location (from non-volatile storage to hardware registers) when the software program is referred to as “implemented in a computer-readable medium”. A processor is considered to be “configured to execute a program” when at least one value associated with the program is stored in a register readable by the processor.

The bus also couples the processor to the network interface device. The interface can include one or more of a modem or network interface. It will be appreciated that a modem or network interface can be considered to be part of the computer system. The interface can include an analog modem, isdn modem, cable modem, token ring interface, satellite transmission interface (e.g. “direct PC”), or other interfaces for coupling a computer system to other computer systems. The interface can include one or more input and/or output devices. The I/O devices can include, by way of example but not limitation, a keyboard, a mouse or other pointing device, disk drives, printers, a scanner, and other input and/or output devices, including a display device. The display device can include, by way of example but not limitation, a cathode ray tube (CRT), liquid crystal display (LCD), or some other applicable known or convenient display device. For simplicity, it is assumed that controllers of any devices not depicted reside in the interface.

In operation, the computer system 600 can be controlled by operating system software that includes a file management system, such as a disk operating system. One example of operating system software with associated file management system software is the family of operating systems known as Windows® from Microsoft Corporation of Redmond, Wash., and their associated file management systems. Another example of operating system software with its associated file management system software is the Linux operating system and its associated file management system. The file management system is typically stored in the non-volatile memory and/or drive unit and causes the processor to execute the various acts required by the operating system to input and output data and to store data in the memory, including storing files on the non-volatile memory and/or drive unit.

Some portions of the detailed description may be presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The algorithms and displays presented herein are not inherently related to any particular computer or other apparatus. Various general-purpose systems may be used with programs in accordance with the teachings herein, or it may prove convenient to construct more specialized apparatus to perform the methods of some embodiments. The required structure for a variety of these systems will appear from the description below. In addition, the techniques are not described with reference to any particular programming language, and various embodiments may, thus, be implemented using a variety of programming languages.

In alternative embodiments, the machine operates as a standalone device or may be connected (e.g., networked) to other machines. In a networked deployment, the machine may operate in the capacity of a server or a client machine in a client-server network environment or as a peer machine in a peer-to-peer (or distributed) network environment.

The machine may be a server computer, a client computer, a personal computer (PC), a tablet PC, a laptop computer, a set-top box (STB), a personal digital assistant (PDA), a cellular telephone, an iPhone, a Blackberry, a processor, a telephone, a web appliance, a network router, switch or bridge, or any machine capable of executing a set of instructions (sequential or otherwise) that specify actions to be taken by that machine.

While the machine-readable medium or machine-readable storage medium is shown in an exemplary embodiment to be a single medium, the term “machine-readable medium” and “machine-readable storage medium” should be taken to include a single medium or multiple media (e.g., a centralized or distributed database, and/or associated caches and servers) that store the one or more sets of instructions. The term “machine-readable medium” and “machine-readable storage medium” shall also be taken to include any medium that is capable of storing, encoding or carrying a set of instructions for execution by the machine and that cause the machine to perform any one or more of the methodologies of the presently disclosed technique and innovation.

In general, the routines executed to implement the embodiments of the disclosure, may be implemented as part of an operating system or a specific application, component, program, object, module or sequence of instructions referred to as “computer programs.” The computer programs typically comprise one or more instructions set at various times in various memory and storage devices in a computer, and that, when read and executed by one or more processing units or processors in a computer, cause the computer to perform operations to execute elements involving the various aspects of the disclosure.

Moreover, while embodiments have been described in the context of fully functioning computers and computer systems, those skilled in the art will appreciate that the various embodiments are capable of being distributed as a program product in a variety of forms, and that the disclosure applies equally regardless of the particular type of machine or computer-readable media used to actually affect the distribution.

Further examples of machine-readable storage media, machine-readable media, or computer-readable (storage) media include but are not limited to recordable type media such as volatile and non-volatile memory devices, floppy and other removable disks, hard disk drives, optical disks (e.g., Compact Disk Read-Only Memory (CD ROMS), Digital Versatile Disks, (DVDs), etc.), among others, and transmission type media such as digital and analog communication links.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize. For example, while processes or blocks are presented in a given order, alternative embodiments may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are, at times, shown as being performed in a series, these processes or blocks may instead be performed in parallel, or may be performed at different times. Further, any specific numbers noted herein are only examples: alternative implementations may employ differing values or ranges.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112, ¶6, other aspects may likewise be embodied as a means-plus-function claim, or in other forms, such as being embodied in a computer-readable medium. (Any claims intended to be treated under 35 U.S.C. § 112, ¶6 will begin with the words “means for”.) Accordingly, the applicant reserves the right to add additional claims after filing the application to pursue such additional claim forms for other aspects of the disclosure.

Although the technology has been described in language that is specific to certain structures and materials, it is to be understood that the invention defined in the appended claims is not necessarily limited to the specific structures and materials described. Rather, the specific aspects are described as forms of implementing the claimed invention. Because many embodiments of the invention can be practiced without departing from the spirit and scope of the invention, the invention resides in the claims hereinafter appended. Unless otherwise indicated, all numbers or expressions, such as those expressing dimensions, physical characteristics, etc. used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques. Moreover, all ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3, 5.8, 9.9994, and so forth).

What is claimed is:

1. A method of verifying the delivery of home care treatment to a patient, comprising
extracting, by a processor, data from an electronic medical billing application including at least a provider name, a location of treatment, a date of treatment, and a patient name;
extracting, by a processor data from a health record application including at least a provider name, a location of treatment, a date of treatment, and a patient name;
obtaining biometric information from the health record application identifying the provider of the treatment;
determining whether the data extracted from the electronic medical billing application matches the data extracted from the health record application; and
verifying the biometric information obtained from the provider of the treatment matches a verification biometric information stored in the health record application for the identified provider.

2. The method of claim 1 wherein the health record application stores at least an encounter audio file from the provider.

3. The method of claim 2 wherein the biometric information obtained from the provider comprises a voice print from the encounter audio file and the verification biometric information is a voice print from a verification audio file.

4. The method of claim 1 further comprising:
verifying the biometric information obtained from a patient during the provision of treatment by the provider matches a verification biometric information stored in the health record application for the patient.

5. The method of claim 1 wherein the biometric information obtained from the patient is a confirmation receipt audio file from which a voice print is obtained and the verification biometric information for the patient is a verification audio file from which a voice print is obtained.

6. The method of claim 1 wherein data extracted from the electronic medical billing application includes a classification code, and the method further comprises determining whether the classification code matches the health record application data.

7. A method of providing an auditable record for the delivery of home care by a provider to a patient, comprising
invoking a health record application on a smart device by a provider of home care;
identifying a provider using a unique identification to access the health record application;
entering data to the health record application via a user interface on the smart device;
determining a date and time that the data is entered into the health record application
determining a location of the smart device using a location-based module; and
verifying the provider with biometric data stored in the health record application and verification biometric data; and
linking the provider identification, date, time, and location to the data stored in the health record application wherein the provider identification, date, time, and location can be verified.

8. The method of claim 7 wherein the location-based module comprises satellite navigation.

9. The method of claim 7 wherein the data entered to the health record application comprises an encounter audio file comprising notes.

10. The method of claim 9 wherein the biometric data comprises a voice print from the encounter audio file and the verification biometric data comprises a voice print from a verification audio file.

11. The method of claim 9 wherein the data entered to the health record application comprises text generated from the encounter audio file.

12. The method of claim 7 wherein the health record application comprises an electronic health record.

13. A method of verifying the delivery of goods or services to a customer, comprising
extracting, by a processor, data from an electronic billing application including at least a provider name, a location of the delivery, a date of the delivery, and a customer name;
extracting, by a processor data from a delivery application including at least a provider name, a location of the delivery, a date of the delivery, and a customer name;
obtaining biometric information from the delivery application identifying the provider of the delivery;
determining whether the data extracted from the electronic billing application matches the data extracted from the delivery application; and
verifying the biometric information obtained from the provider of the delivery matches a verification biometric information stored in the delivery application for the identified provider.
